(19) United States
(12) Patent Application Publication
(10) Pub. No.: US 2019/0388944 A1
(43) Pub. Date: Dec. 26, 2019

(54) Method and System for Control of Steel Strip Microstructure in Thermal Processing Equipment Using Electro Magnetic Sensors

(71) Applicant: PRIMETALS TECHNOLOGIES USA LLC, ALPHARETTA, GA (US)

(72) Inventors: William T. Larsen, Lexington, MA (US); John G. Stedge, Chalfont, PA (US)

(73) Assignee: PRIMETALS TECHNOLOGIES USA LLC, ALPHARETTA, GA (US)

(21) Appl. No.: 16/259,048

(22) Filed: Jan. 28, 2019

Related U.S. Application Data
(60) Provisional application No. 62/688,081, filed on Jun. 21, 2018.

(51) Int. Cl. B21B 37/74; B21B 38/00; C21D 11/00

(52) U.S. Cl. CPC B21B 37/74; B21B 38/006; G01N 33/20; B21B 2201/02; C21D 11/005

(57) ABSTRACT

A steel strip processing system is provided that includes a plurality of microstructure sensors that measure the phase fraction in a steel strip at desired locations in a processing furnace. A process control system includes a plurality of control loops for receiving the outputs of the microstructure sensors to determine the amount of heating and cooling required to achieve a desired phase fraction at the desired locations in the processing furnace. One or more energy systems that receive the output of the process control system to coordinate the heating or cooling of the desired locations to achieve the desired phase fraction.

### PRIORITY INFORMATION

This application claims priority from provisional application Ser. No. 62/688,081 filed Jun. 21, 2018, which is incorporated herein by reference in its entirety.

### BACKGROUND OF THE INVENTION

The invention relates to the field of steel strip processing, and in particular to steel strip microstructures in thermal processing using electromagnetic sensors.

During production processing of metals, such as steel, rolling of the metal is followed by controlled cooling. During the production processing, particularly the cooling process, a microstructure of the metal evolves and results in a final microstructure of the processed metal. The microstructure of the processed metal has an impact on many aspects of the metal's character, such as tensile strength.

Conventional microstructural analysis techniques are destructive and involve removing samples for analysis from, for example, the end of a coil of the processed material. This is time-consuming, costly, does not allow continuous monitoring, and assesses only a small fraction of the material processed.

When the processed material is steel, it is known that 35 electromagnetic techniques can monitor steel phase transformations by detecting the ferromagnetic phase change due to the changes in electrical conductivity and magnetic permeability within the steel. Furthermore, if a coil is placed in the vicinity of the steel being processed, this results in a change in 40 impedance measurements for the coil because conductivity and permeability are influenced by the steel's micro structure. For example austenite, the stable phase of iron at elevated temperatures, is paramagnetic whereas the stable low temperature phases ferrite, pearlite, bainite and martensite are 45 ferromagnetic below the Curie temperature of about 7 60° C. Steel properties vary strongly with the volume fractions of these phases, which are controlled largely by the cooling rate and alloy content of the steel.

### SUMMARY OF THE INVENTION

According to one aspect of the invention, there is provided a steel strip processing system. The steel strip processing system includes a plurality of microstructure sensors that measure the phase fraction in a steel strip at desired locations in a processing furnace. A process control system includes a plurality of control loops for receiving the outputs of the microstructure sensors to determine the amount of heating and cooling required to achieve a desired phase fraction at the desired locations in the processing furnace. One or more energy systems that receive the output of the process control system to coordinate the heating or cooling of the desired locations to achieve the desired phase fraction.

According to another aspect of the invention, there is provided a method of steel strip thermal processing. The method includes measuring the phase fraction in a steel strip at desired locations in a processing furnace using a plurality of microstructure sensors. Also, the method includes providing a process control system that includes a plurality of control loops for receiving the outputs of the microstructure sensors to determine the amount of heating and cooling required to achieve a desired phase fraction at the desired locations in the processing furnace. Furthermore, the method includes coordinating the heating or cooling of the desired locations to achieve the desired phase fraction using one or more energy systems that receive the output of the process control system.

### BRIEF DESCRIPTION OF THE DRAWINGS

FIG. 1 is a schematic diagram illustrating a process control system used in accordance with the invention; and

FIG. 2 is a schematic diagram illustrating another embodiment of the process control system used in accordance with the invention.

### DETAILED DESCRIPTION OF THE INVENTION

The invention provides a system and method to control the thermal processing of advanced high strength steels in a continuous galvanizing line or continuous annealing line. In order for the steel producer to create a steel with the desired properties, they must be able to control the phase fraction that is associated with the amount of ferrite vs. austenite during inter-critical annealing including heating and holding to a temperature between the AC1 eutectoid transformation temperature and the AC3 full austenite transformation temperature. One must also control the amount of retained austenite (vs. martensite or other ferrite phases) during the subsequent cooling process. Controlling the extent of the transformations during the thermal processing of the steel is necessary to achieve the desired final microstructure for a given steel composition

The invention involves implementing an electromagnetic sensor designed to directly measure the phase fraction in a steel strip at appropriate locations in a processing furnace and using the output from the sensors to control, in whole or in part, the amount of heating and cooling to achieve the desired phase fraction at the desired location in the processing furnace. Moreover, the invention utilizes an additional electromagnetic sensor at or near the end of a cooling section of a thermal processing furnace for the purpose of controlling the amount of cooling. At each location, the signal from the sensor measuring phase fraction will be used as the input to a controller which is used to control the amount of heating or cooling respectively.

The control loops could be used in a direct closed loop where the signal from the electromagnetic sensor is used to directly control the heating or cooling (example burner firing rate or induction coil power output for heating or fan speed for convection cooling). Alternatively one could use nested closed control loops where the output from the electromagnetic sensor is used as an input to a closed loop controller whose output is a metal temperature set point. This temperature set point is then used as the input to a separate temperature controller which used in conjunction with a strip temperature measurement sensor to control the amount of heating or cooling.

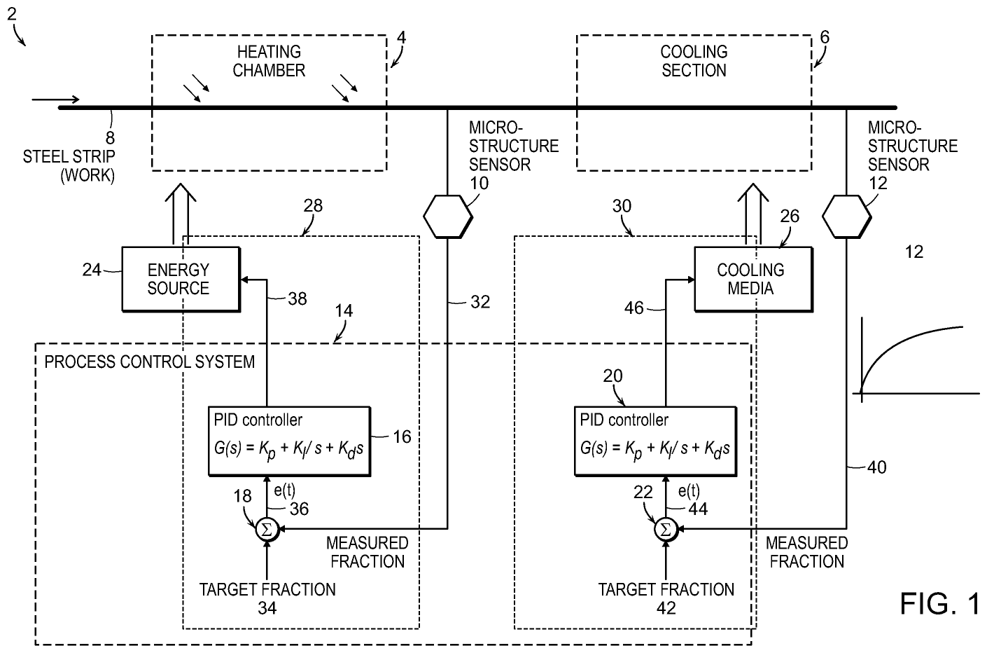

FIG. 1 is a schematic diagram illustrating a steel strip processing system 2 used in accordance with the invention. In a rolling mill, a steel strip 8 is presented to a heating chamber 4 used in the annealing process. A cooling section 6 is provided for cooling the steel strip 8 after being annealed. A first microstructure sensor 10 is positioned at the output of the heating chamber 4, and a second microstructure sensor 12 is positioned at the output of the cooling section 6. The first and second microstructure sensors 10, 12 both the measure the phase fraction in a steel strip 8 at their appropriate locations. The results of the measured phase fraction of the first microstructure 10 as well as the second microstructure 12 are sent to a process control system 14.

The process control system 14 includes two control loops 28, 30 used for controlling the temperatures in both the heating chamber 4 and cooling section 6. The first control loop 28 includes a first summation module 18 and a first PID controller 16 having a specified transfer function. The second control loop 30 includes a second summation module 22 and a second PID controller 20 having a specified transfer function.

The first summation module 18 receives as input the output 32 of the first microstructure sensor 10 and a target fraction 34. The output 36 of the first summation module 18 is provided to the first PID controller 16. The first PID controller 16 provides an output 38, in accordance with its transfer function, to an energy source 24 to control the heating temperature of the heating chamber 4. The second summation module 22 receives as input the output 40 of the second microstructure sensor 12 and a target fraction 42. The output 44 of the second summation module 22 is provided to the second PID controller 20. The second PID controller 20 provides an output 46, in accordance with its transfer function, to a cooling media 26 to control the cooling temperature of the cooling section 6.

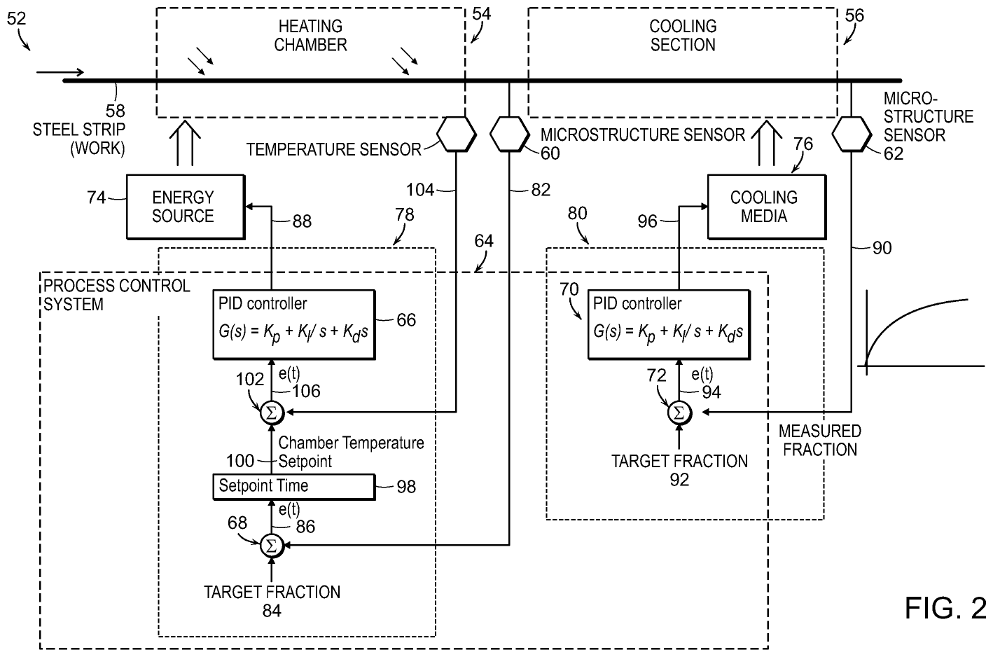

FIG. 2 is a schematic diagram illustrating another embodiment of the strip steel processing system 52 used in accordance with the invention. In a rolling mill, a steel strip 58 is presented to a heating chamber 54 used in the annealing process. A cooling section 56 is provided for cooling the steel strip 58 after being annealed. A first microstructure sensor 60 is positioned at the output of the heating chamber 54, and a second microstructure sensor 62 is positioned at the output of the cooling chamber 56. The first and second microstructure sensors 60, 62 both measure the phase fraction in a steel strip at their appropriate locations. The results of the measured phase fraction of the first microstructure 60 as well as the second microstructure 62 are sent to a process control system 64.

The process control system 64 includes two control loops 78, 80 used for controlling the temperatures in both the heating chamber 54 and cooling section 56. The first control loop 78 includes a first summation module 68, a PID controller 66, and a set point trim module 98 that receives the output of the first summation module 86. The set point trim module 98 is a controller whose output 100 is a metal temperature set point. A second summation module 102 receives the output 100 of the set point trim module 98 and the output 104 of a temperature sensor 106 positioned on the heating chamber 54. A first PID controller 66 is provided having a specified transfer function. The output 106 of the second summation module 102 is provided to the first PID controller 66. The first PID controller 66 provides its output 88, in accordance with its transfer function, to an energy source 74 to control the heating temperature of the heating chamber 54.

The second control loop 80 includes a third summation module 72 and a second PID controller 70 having a specified transfer function. The third summation module 72 receives as input the output 90 of the second microstructure sensor 62 and a target fraction 92. The output of the third summation module is provided to the second PID controller 70. The second PID controller 70 provides an output 96, in accordance with its transfer function, to a cooling media 76 to control the temperature of the cooling section 56.

The first microstructure sensor and second microstructure sensor shown in FIGS. 1 and 2 comprise electromagnetic sensors. One of the keys to using electromagnetic sensors they can directly measure the phase fraction of the steel microstructure. In previous solutions, only the temperature is measured which is used as a proxy to achieve the desired phase fraction of the steel. Temperature measurements are typically taken using non-contact radiation detectors. This type of detector can be inaccurate when measuring advanced high strength steels because of changes in surface emissivity which must be known in order to achieve an accurate reading. In addition, the required processing temperatures are typically determined in a lab environment which may not be fully representative of the production environment. By measuring the phase fraction directly the processing temperatures (heating and cooling) can be adjusted automatically.

Finally, the phase fraction properties at the proposed measuring locations are interim points in the thermal processing of the final materials. The final microstructure of the steel at the end of the process will be different. If the desired metal properties are not achieved as desired it can be difficult to determine which interim temperature to change with the previous solution. This is often achieved by trial and error. With the proposed invention it will be much easier to achieve the desired final microstructure and properties of the steel being processed.

Although the present invention has been shown and described with respect to several preferred embodiments thereof, various changes, omissions and additions to the form and detail thereof, may be made therein, without departing from the spirit and scope of the invention.

What is claimed is:

1. A steel strip processing system comprising:
a plurality of microstructure sensors that measure the phase fraction in a steel strip at desired locations in a processing furnace;
a process control system that includes a plurality of control loops for receiving the outputs of the microstructure sensors to determine the amount of heating and cooling required to achieve a desired phase fraction at the desired locations in the processing furnace; and
one or more energy systems that receive the output of the process control system to coordinate the heating or cooling of the desired locations to achieve the desired phase fraction.

2. The steel strip processing system of claim 1, wherein the microstructure sensors comprise electromagnetic sensors.

3. The steel strip processing system of claim 1, wherein the processing furnace comprises a heating chamber for heating or annealing the steel strip.

4. The steel strip processing system of claim 1, wherein the processing furnace comprises a cooling section for cooling the steel strip.

5. The steel strip processing system of claim 3, wherein one of the control loops provides the parameters used for defining the temperature used for the steel strip the heating chamber.

6. The steel strip processing system of claim 4, wherein one of the control loops provides the parameters used for defining the temperature for cooling the steel strip in the cool section.

7. The steel strip processing system of claim 2, wherein the electromagnetic sensors directly measure the phase fraction of the steel strip.

8. The steel strip processing system of claim 1, wherein one of the microstructure sensors is positioned at the end of the heating chamber.

9. The steel strip processing system of claim 1, wherein one of the microstructure sensors is positioned at the end of the cooling section.

10. The steel strip processing system of claim 1, wherein one of the control loops comprises a set point trim module that defines a metal temperature set point.
A method of steel strip thermal processing comprising:
measuring the phase fraction in a steel strip at desired locations in a processing furnace using a plurality of microstructure sensors;
providing a process control system that includes a plurality of control loops for receiving the outputs of the microstructure sensors to determine the amount of heating and cooling required to achieve a desired phase fraction at the desired locations in the processing furnace; and

11. Coordinating heating or cooling of the desired locations to achieve the desired phase fraction using one or more energy systems that receives the output of the process control system.

12. The method of claim 11, wherein the microstructure sensors comprise electromagnetic sensors.

13. The method of claim 11, wherein the processing furnace comprises a heating chamber for heating or annealing the steel strip.

14. The method of claim 11, wherein the processing furnace comprises a cooling section for cooling the steel strip.

15. The method of claim 13, wherein one of the control loops provides the parameters used for defining the temperature used for the steel strip the heating chamber.

16. The method of claim 14, wherein one of the control loops provides the parameters used for defining the temperature for cooling the steel strip in the cool section.

17. The method of claim 12, wherein the electromagnetic sensors directly measure the phase fraction of the steel strip.

18. The method of claim 11, wherein one of the microstructure sensors is positioned at the end of the heating chamber.

19. The method of claim 11, wherein one of the microstructure sensors is positioned at the end of the cooling section.

20. The method of claim 11, wherein one of the control loops comprises a set point trim module that defines a metal temperature set point.
